(19) United States
(12) Patent Application Publication
(10) Pub. No.: US 2017/0319432 A1
(43) Pub. Date: Nov. 9, 2017

(54) PRESS NEEDLES WITH AN ALIGNMENT STRUCTURE

(71) Applicants: Wu-Chi HO, Taipei City (TW); Hsin-Ying Ho, Taipei City (TW)

(72) Inventors: Wu-Chi HO, Taipei City (TW); Hsin-Ying Ho, Taipei City (TW)

(21) Appl. No.: 15/582,565

(22) Filed: Apr. 28, 2017

(30) Foreign Application Priority Data
May 5, 2016 (TW) 105113910

(51) Int. Cl. A61H 39/08; A61H 39/00; A61B 17/34

(52) U.S. Cl. CPC A61H 39/086; A61B 17/3403; A61H 39/002

(57) ABSTRACT

Provided is an press needles with an alignment structure having a conductor and multiple needles. The conductor having two opposite ends has an acupuncture area and a holding area. The acupuncture area is located at one end of the conductor, and the holding area is located at the other end of the conductor. The needles are sequentially aligned at a side of the conductor. Therefore, the alignment structure offers the patient better treatments and decreasing the period of treatment.

### CROSS REFERENCE

This application claims priority to TW Patent Application Serial No. 105113910, filed on May 5, 2016, the content of which is hereby incorporated by reference in its entirety.

### BACKGROUND OF THE INVENTION

### 1. Field of the Invention

The present invention relates to press needles, particularly to an press needles with an alignment structure.

### 2. Description of the Prior Arts

Acupuncture is traditional Chinese medicine derived from the wisdom of the ancients, and the acupoints on a human body distribute at the circulation paths of the organ meridians. Doctors can use acupuncture needles to perform acupuncture on the acupoints of the patient for pain relief.

The ear has a lot of ear acupoints that can be used to remove inflammation, repair body tissues, activate mechanisms, improve immunity, and prevent from diseases. The distance between two ear acupoints is one twentieth of the distance between two body acupoints. For instance, the distance between two body acupoints is less than 3.5 centimeters (cm), so the distance between two ear acupoints is less than 0.175 cm. Some of the adjacent acupoints are aligned as a meridian, especially the conception vessel (ren mai), governing vessel (du mai), and bladder meridian.

In addition to the plum blossom needle that is constituted of a circular arrangement of multiple needles for bloodletting, other types of acupuncture needles are each composed of one single needle such as the press needle, intradermal embedding needle, long needle, etc. As shown in FIG. 7, the press needle 90 is the most common needle for treatment through ear acupoints. The press needle 90 has a circular base 91 and a tip needle 92 connected to the circular base 91. However, using the single needle such as the press needle 90 for acupuncture is slow and inconvenient, the circular base 91 is too small to hold and press, the tip needle 92 is easily twisted, and the press needle 90 is hard to be electrically connected for electro-acupuncture treatment. Besides, the acupoints in the curved structure of the ear are hard to be acupunctured by the press needle 90, especially the acupoints in the concave site of the ear are difficult to be acupunctured by the second press needle 90.

Therefore, the disadvantages in the prior arts should be resolved.

### SUMMARY OF THE INVENTION

According to the above description, the objective of the present invention is to provide an press needles with an alignment structure for offering the patient better treatments and decreasing the period of treatment.

To achieve the above objective of the present invention, the invention provides the press needles with an alignment structure comprising a conductor and multiple needles. The conductor having two opposite ends comprises an acupuncture area and a holding area. The acupuncture area is located at one of the two opposite ends of the conductor, and the holding area is located at the other end of the conductor. The needles are sequentially aligned at a side of the conductor.

Preferably, the conductor is formed as a sheet.

More preferably, the conductor has two opposite surfaces and an axial direction of each of the needles is perpendicular to one of the two opposite surfaces of the conductor.

More preferably, an axial direction of each of the needles is perpendicular to an axial direction of the conductor, and the axial direction of each of the needles and one of the two opposite surfaces of the conductor are at the same plane.

Preferably, the needles include five needles.

Preferably, the press needles with an alignment structure further comprises a wire electrically connected to the holding area of the conductor.

Preferably, the press needles with an alignment structure further comprises a tape attached to the conductor.

The advantage of the press needles with an alignment structure of the present invention is that multiple ear acupoints can be acupunctured by the needles formed on the conductor, such that the acupuncture speed can be accelerated and the stability of the press needles with an alignment structure on the ear can be enhanced. The holding area of the conductor is large and facilitates ease in holding, such that the needles will not tend to twist during acupuncture. Besides, the holding area is easily clamped by the clamp of the wire for electro-acupuncture, thereby saving the treatment time, enhancing treatment effect, and reducing treatment period. Particularly, this treatment provides benefits with inflammation reduction, repair, mechanism, immunity, and disease prevention.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of an press needles with an alignment structure of the present invention;

FIG. 2 is a perspective view of a preferred embodiment of the press needles with an alignment structure of the present invention;

FIG. 3 is illustrates an operational view of the press needles with an alignment structure of the present invention, shown with a wire;

FIG. 4 is illustrates an operational view of the press needles with an alignment structure of the present invention, shown bended;

FIG. 5 is an operational view of the press needles with an alignment structure of the present invention, shown with a wire;

FIG. 6 is an operational view of the press needles with an alignment structure of the present invention, shown having a tape;

FIG. 7 is the press needle of the prior art.

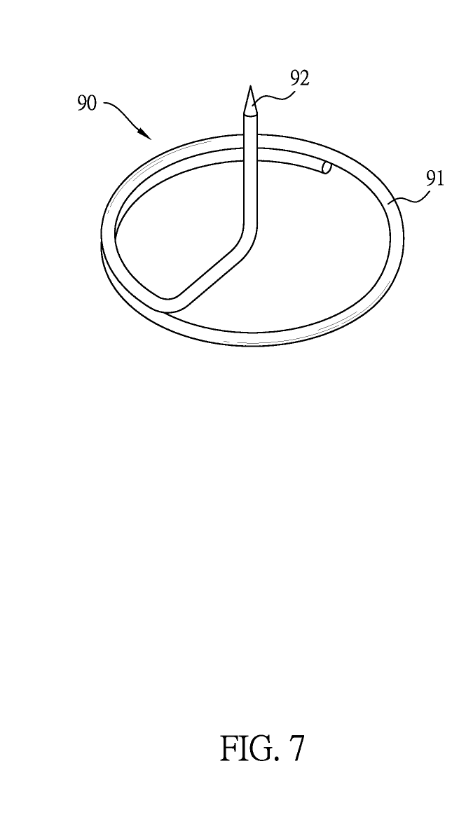

### DETAILED DESCRIPTION OF THE PREFERRED EMBODIMENTS

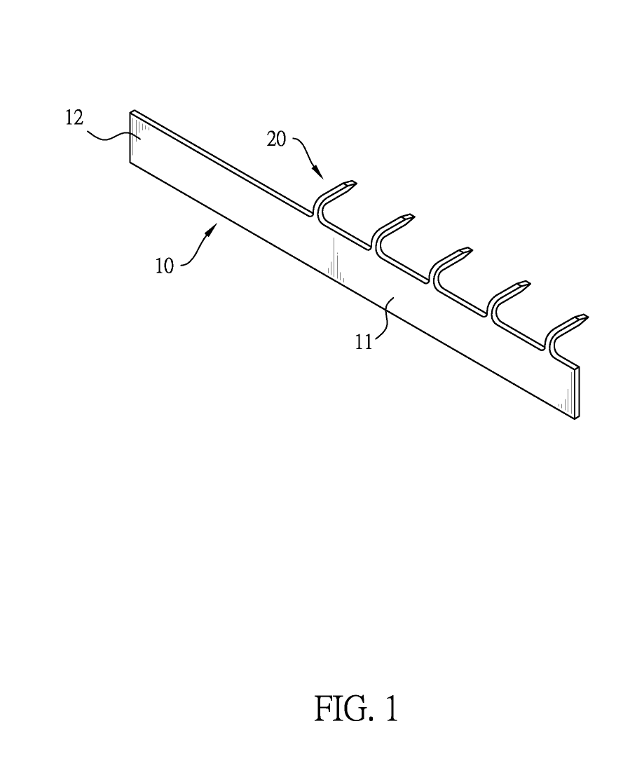

As shown in FIG. 1, the first preferred embodiment of the present invention provides an press needles with an alignment structure, the alignment structure comprising a conductor 10 and multiple needles 20.

The conductor 10 has two opposite ends, and the material of the conductor 10 includes, but is not limited to, stainless steel. The conductor 10 is formed as a sheet, and comprises an acupuncture area 11 and a holding area 12. The acupuncture area 11 is located at one of the two opposite ends of the conductor 10; the holding area 12 is located at the other end of the conductor 10.

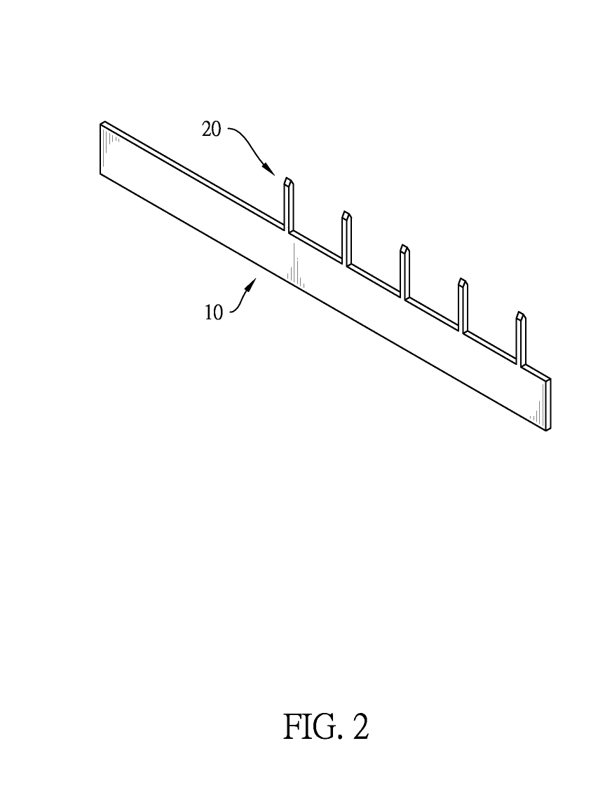

The multiple needles 20 are five needles 20 sequentially aligned at a side of the conductor 10. The conductor 10 has two opposite surfaces, and an axial direction of each of the needles 20 is perpendicular to one of the two opposite surfaces of the conductor 10. As shown in FIG. 2, in a preferred embodiment, an axial direction of each of the needles 20 is perpendicular to an axial direction of the conductor 10, and the axial direction of each of the needles 20 and one of the two opposite surfaces of the conductor 20 are at the same plane.

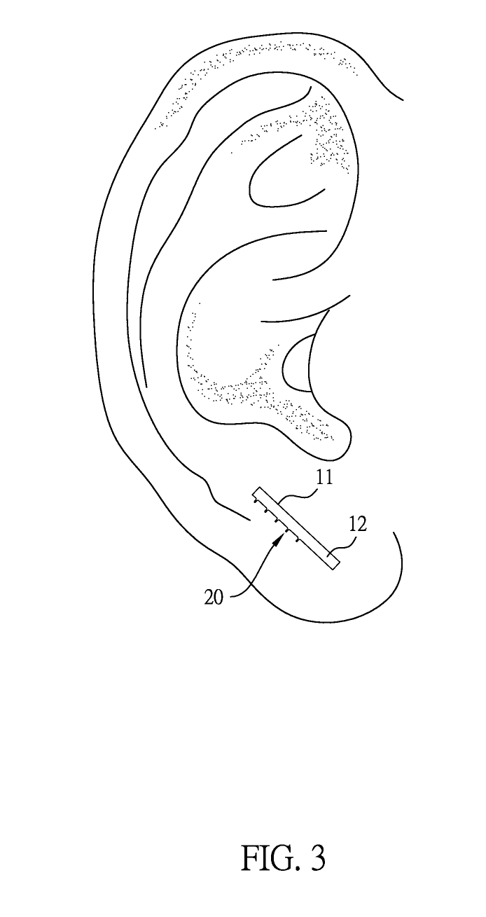

As shown in FIG. 3, when the present invention is in use, the holding area 12 is held by a user's hand so the acupoints are easily acupunctured by the needles 20 of the acupuncture area 11. The needles 20 sequentially aligned are corresponding to adjacent acupoints which are aligned as a meridian. As the press needles with an alignment structure of the present invention having five needles 20 can acupuncture five ear acupoints at the same time and its speed of acupuncture is faster than that by using the traditional single needle. Besides, the needles 20 sequentially aligned can easily acupuncture the acupoints in such small distances in the ear. Even if the target acupoints are not accurately acupunctured by the needles 20, as long as adjacent acupoints are acupunctured, it still has some auxiliary therapeutic effect because the position of meridian is right, and there is no need to acupuncture again. Therefore, ear acupoints can be easily acupunctured at one time by the press needles with an alignment structure of the present invention, and the press needles with an alignment structure is user friendly for use by the patients themselves.

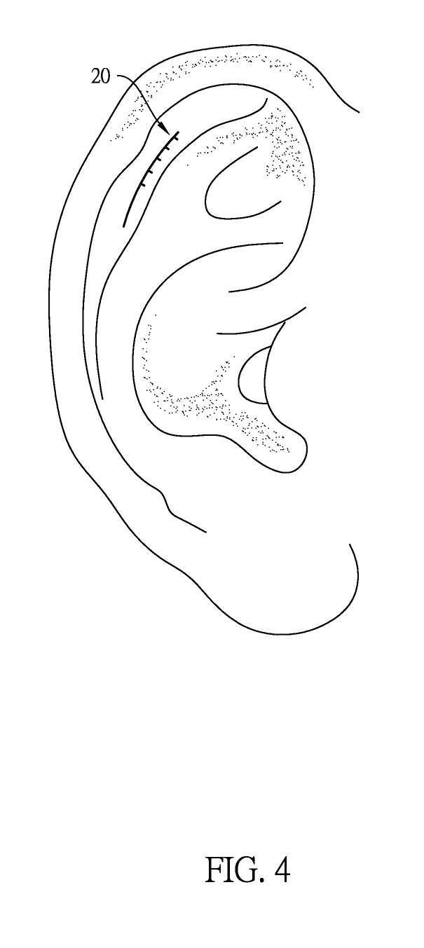

As shown in FIG. 4, in a preferred embodiment, the press needles with an alignment structure can be bended to fit the acupoints in the curved structure of the ear, such that the acupoints can be easily acupunctured by the needles 20 of the press needles with an alignment structure.

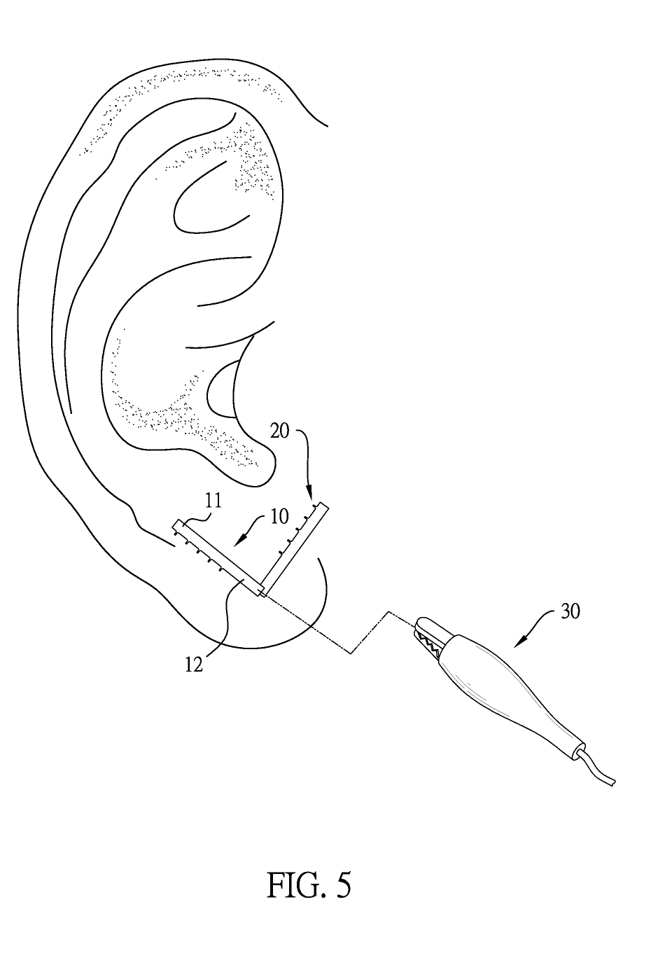

As shown in FIG. 5, in a preferred embodiment, the press needles with an alignment structure further comprises a wire 30 electrically connected to the holding area 12 of the conductor 10. In a preferred embodiment, the wire 30 has a clamp located at an end of the wire, and the holding area 12 of the conductor 10 is clamped by the clamp. For example, the traditional treatment of facial hemiplegia is acupuncturing the face by individual single needles, but the treatment of facial hemiplegia by the press needles with an alignment structure of the present invention only needs to acupuncture the ear acupoints. The cheek area meridian of ear acupoints is acupunctured by the acupuncture area 11 of the conductor 10. Besides, the press needles with an alignment structure of the present invention can use electro-acupuncture treatment through the wire 30, such that the therapeutic effect will be greater than acupuncture only. Furthermore, each of the holding areas 12 of the conductors 10 of two alignment structures of press needles are overlapped at the desired angle, and the target ear acupoints are acupunctured by the needles 20 of the two alignment structures of press needles.

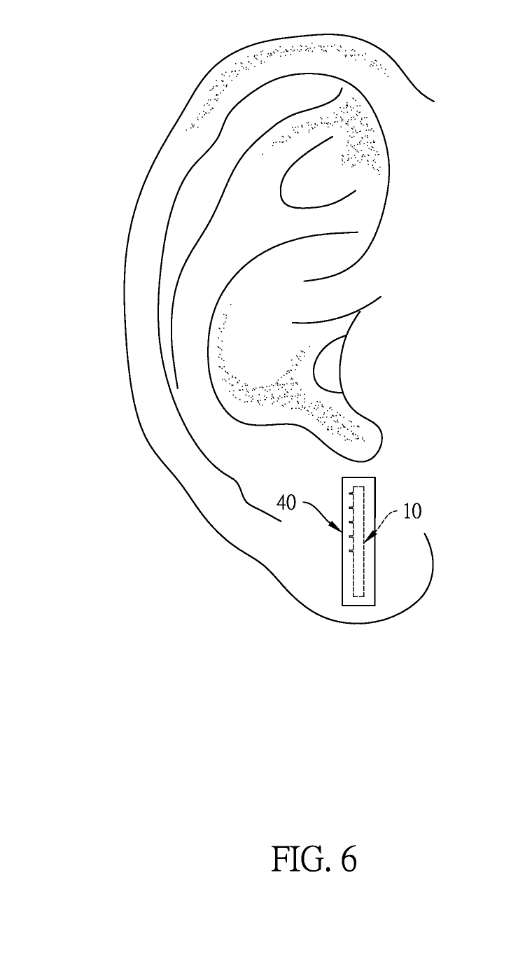

As shown in FIG. 6, in another preferred embodiment, the press needles with an alignment structure of the present invention further comprises a tape 40 attached to one of the two opposite surfaces of the conductor 10. When ear acupoints are acupunctured by the press needles with an alignment structure of the present invention, the tape 40 is further attached to the conductor 10 to enhance the stability of the press needles with an alignment structure on the ear.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An press needles with an alignment structure, comprising:
a conductor having two opposite ends, the conductor comprising:
an acupuncture area located at one of the two opposite ends of the conductor; and
a holding area located at the other end of the conductor; and
multiple needles sequentially aligned at a side of the conductor.

2. The press needles with an alignment structure as claimed in claim 1, wherein the conductor is formed as a sheet.

3. The press needles with an alignment structure as claimed in claim 2, wherein the conductor has two opposite surfaces, and an axial direction of each of the needles is perpendicular to one of the two opposite surfaces of the conductor.

4. The press needles with an alignment structure as claimed in claim 2, wherein the conductor has two opposite surfaces, and an axial direction of each of the needles is perpendicular to an axial direction of the conductor, and the axial direction of each of the needles and one of the two opposite surfaces of the conductor are at the same plane.

5. The press needles with an alignment structure as claimed in claim 1, wherein the needles include five needles.

6. The press needles with an alignment structure as claimed in claim 2, wherein the needles include five needles.

7. The press needles with an alignment structure as claimed in claim 1, further comprising a wire electrically connected to the holding area of the conductor.

8. The press needles with an alignment structure as claimed in claim 2, further comprising a wire electrically connected to the holding area of the conductor.

9. The press needles with an alignment structure as claimed in claim 1, further comprising a tape attached to the conductor.

10. The press needles with an alignment structure as claimed in claim 2, further comprising a tape attached to one of the two opposite surfaces of the conductor.
